(19) United States
(12) Patent Application Publication
(10) Pub. No.: US 2021/0313028 A1
(43) Pub. Date: Oct. 7, 2021

(54) PHYSIOLOGICAL MONITORING SYSTEM

(71) Applicant: Medtronic, Inc., Minneapolis, MN (US)

(72) Inventors: Scott A. Hareland, Lino Lakes, MN (US); Fabrice Navers, Edina, MN (US)

(21) Appl. No.: 17/166,302

(22) Filed: Feb. 3, 2021

Related U.S. Application Data
(60) Provisional application No. 63/003,343, filed on Apr. 1, 2020.

(51) Int. Cl. G16H 15/00; A61B 18/14; A61B 18/02; G16H 20/30; G16H 20/40; G16H 50/70

(52) U.S. Cl. CPC G16H 15/00; A61B 18/1492; A61B 18/02; A61B 2017/00115; G16H 20/40; G16H 50/70; G16H 20/30

(57) ABSTRACT

A method of monitoring a patient for phrenic nerve collateral damage during a cardiac ablation procedure. The method includes measuring at least one from the group consisting of compound motor action potential (CMAP) and accelerometer signals in response to stimulating of the phrenic nerve. Real-time data is displayed on a display, the real-time data including the at least one from the group consisting of the measured CMAP and accelerometer signals. Long-term trend data is simultaneously displayed on the display, the long-term trend data being associated with the measured at least one from the group consisting of CMAP and accelerometer signals.

### CROSS-REFERENCE TO RELATED APPLICATION

This application claims the benefit of U.S. Application Ser. No. 63/003,343 filed Apr. 1, 2020.

### FIELD

The present technology is generally related to a method of monitoring a patient for phrenic nerve collateral damage during a cardiac ablation procedure.

### BACKGROUND

Physiological monitoring of patients is often required during medical procedures. During cardiac ablation, particularly cryoablation of heart tissue, collateral damage of non-cardiac tissues, such as the phrenic nerve, may be damaged. Methods in use today involve pacing the superior phrenic nerve (PN) and manually checking for patient diaphragmatic stimulation. More sophisticated methods include the PN stimulation but utilize muscular contraction electrical signals, e.g. CMAP, or accelerometers on the patient to quantify the response to the stimulation. When a reduction in the patient's diaphragmatic response is detected, a clinician can choose to alter or end the therapy in order to preserve the non-cardiac tissue/nerve before permanent damage occurs.

In a typical CMAP, accelerometer, or other physiological monitoring system, appropriate display and analysis of information can be crucial for prompt and effective clinical response. Too little or too much data can overwhelm the clinician and lead to false-positive results or delayed action, either of which can be disruptive and/or harmful.

### SUMMARY

The techniques of this disclosure generally relate to a method of monitoring a patient for phrenic nerve collateral damage during a cardiac ablation procedure.

In one aspect, the present disclosure provides a method of monitoring a patient for phrenic nerve collateral damage during a cardiac ablation procedure. The method includes measuring at least one from the group consisting of compound motor action potential (CMAP) and accelerometer signals in response to stimulating of the phrenic nerve. Real-time data is displayed on a display, the real-time data including the at least one from the group consisting of the measured CMAP and accelerometer signals. Long-term trend data is simultaneously displayed on the display, the long-term trend data being associated with the measured at least one from the group consisting of CMAP and accelerometer signals.

In another aspect of this embodiment, displaying the real-time data includes displaying a rolling window of the real time data.

In another aspect of this embodiment, the rolling widow includes a predetermined number of previous cycles of phrenic nerve stimulation.

In another aspect of this embodiment, the real-time data is filtered before it is displayed on the display.

In another aspect of this embodiment, the real-time data is superimposed with predetermined signal feature extraction markers.

In another aspect of this embodiment, the predetermined signal feature extraction markers are color coded.

In another aspect of this embodiment, the predetermined signal feature extraction markers are correlated to a predetermined percentage threshold from peak amplitude.

In another aspect of this embodiment, displaying the real-time data further includes displaying a pre-ablation baseline peak amplitude.

In another aspect of this embodiment, simultaneously displaying the long-term trend data includes displaying a peak amplitude from each of a previous cycle of phrenic nerve stimulation.

In another aspect of this embodiment, each peak amplitude is color coded.

In another aspect of this embodiment, each color-coded peak amplitude is correlated to a predetermined percentage threshold from a baseline peak amplitude.

In another aspect of this embodiment, the method further includes displaying with the long-term trend data a point at which the cardiac ablation procedure is initiated.

In one aspect, a method of monitoring a patient for phrenic nerve collateral damage during a cardiac ablation procedure includes stimulating the phrenic nerve. A compound motor action potential (CMAP) signal is measured in response to the stimulation of the phrenic nerve. A rolling window of real-time data including a predetermined number of previous cycles of phrenic nerve stimulation is displayed on a display, the real-time data including the measured CMAP signal. Long-term trend data is simultaneously displayed on the display, the long-term trend data being associated with the measured CMAP signal and including a peak amplitude from each of the previous cycles of phrenic nerve stimulation.

In another aspect of this embodiment, the real-time data is superimposed with predetermined signal feature extraction markers.

In another aspect of this embodiment, the predetermined signal feature extraction markers are color-coded.

In another aspect of this embodiment, the predetermined signal feature extraction markers are correlated to a predetermined percentage threshold from peak amplitude.

In another aspect of this embodiment, displaying the real-time data further includes displaying a pre-ablation baseline peak amplitude.

In another aspect of this embodiment, each peak amplitude is color-coded, and wherein each color-coded peak amplitude is correlated to a predetermined percentage threshold from the pre-ablation baseline peak amplitude.

In another aspect of this embodiment, the method further includes displaying with the long-term trend data a point at which the cardiac ablation procedure is initiated.

In one aspect, a method of monitoring a patient for phrenic nerve collateral damage during a cardiac ablation procedure includes stimulating the phrenic nerve. A compound motor action potential (CMAP) signal is measured in response to the stimulation of the phrenic nerve. A rolling window of real-time data including a predetermined number of previous cycles of phrenic nerve stimulation is displayed on a display, the real time data including the measured CMAP signal and a pre-ablation baseline peak amplitude. Color coded predetermined signal feature extraction markers are superimposed on the real-time data. Long term trend data is simultaneously displayed on the display, the long-term trend data being associated with the measured CMAP signal and including a peak amplitude from each of the previous cycles of phrenic nerve stimulation, each peak amplitude is color-coded, and each color-coded peak amplitude is correlated to a predetermined percentage threshold from the pre-ablation baseline peak amplitude.

The details of one or more aspects of the disclosure are set forth in the accompanying drawings and the description below. Other features, objects, and advantages of the techniques described in this disclosure will be apparent from the description and drawings, and from the claims.

### BRIEF DESCRIPTION OF THE DRAWINGS

A more complete understanding of the present invention, and the attendant advantages and features thereof, will be more readily understood by reference to the following detailed description when considered in conjunction with the accompanying drawings wherein:

FIG. 1 is an assembly view of an electrosurgical medical system constructed in accordance with the principles of the present application;

FIG. 2 is a front view of a displaying showing a combination of real-time data and long-term trend data; and

FIG. 3 is a flow chart showing an exemplary method of the present application.

### DETAILED DESCRIPTION

It should be understood that various aspects disclosed herein may be combined in different combinations than the combinations specifically presented in the description and accompanying drawings. It should also be understood that, depending on the example, certain acts or events of any of the processes or methods described herein may be performed in a different sequence, may be added, merged, or left out altogether (e.g., all described acts or events may not be necessary to carry out the techniques). In addition, while certain aspects of this disclosure are described as being performed by a single module or unit for purposes of clarity, it should be understood that the techniques of this disclosure may be performed by a combination of units or modules associated with, for example, a medical device.

In one or more examples, the described techniques may be implemented in hardware, software, firmware, or any combination thereof. If implemented in software, the functions may be stored as one or more instructions or code on a computer-readable medium and executed by a hardware-based processing unit. Computer-readable media may include non-transitory computer-readable media, which corresponds to a tangible medium such as data storage media (e.g., RAM, ROM, EEPROM, flash memory, or any other medium that can be used to store desired program code in the form of instructions or data structures and that can be accessed by a computer).

Instructions may be executed by one or more processors, such as one or more digital signal processors (DSPs), general purpose microprocessors, application specific integrated circuits (ASICs), field programmable logic arrays (FPGAs), or other equivalent integrated or discrete logic circuitry. Accordingly, the term “processor” as used herein may refer to any of the foregoing structure or any other physical structure suitable for implementation of the described techniques. Also, the techniques could be fully implemented in one or more circuits or logic elements.

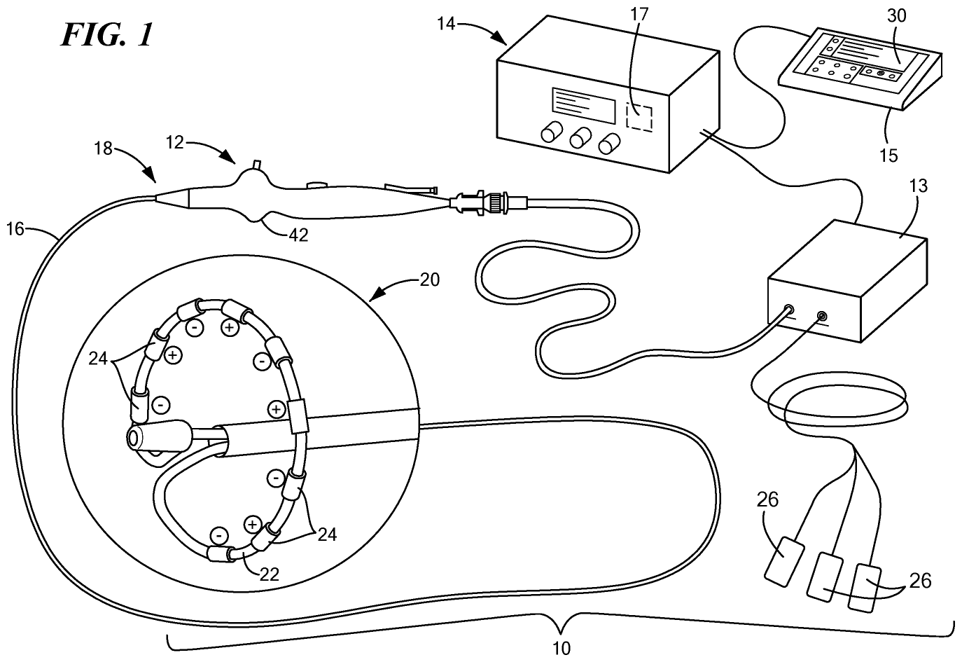

Referring now to the drawing figures in which like reference designations refer to like elements, an embodiment of a medical system constructed in accordance with principles of the present invention is shown in FIG. 1 and generally designated as “10.” The system 10 generally includes a medical device 12 that may be coupled directly to an energy supply, for example, a generator 14 including an energy control, delivering and monitoring system or indirectly through a catheter electrode distribution system 13. A remote controller 15 may further be included in communication with the generator for operating and controlling the various functions of the generator 14. The medical device 12 may generally include one or more diagnostic or treatment regions for energetic, therapeutic and/or investigatory interaction between the medical device 12 and a treatment site. The treatment region(s) may deliver, for example, radiofrequency ablation, cryoablation, or pulsed electroporation energy to a tissue area in proximity to the treatment region(s).

The medical device 12 may include an elongate body 16 passable through a patient's vasculature and/or positionable proximate to a tissue region for diagnosis or treatment, such as a catheter, sheath, or intravascular introducer. The elongate body 16 may define a proximal portion 18 and a distal portion 20, and may further include one or more lumens disposed within the elongate body 16 thereby providing mechanical, electrical, and/or fluid communication between the proximal portion of the elongate body 16 and the distal portion of the elongate body 16. The distal portion 20 may generally define the one or more treatment region(s) of the medical device 12 that are operable to monitor, diagnose, and/or treat a portion of a patient. The treatment region(s) may have a variety of configurations to facilitate such operation. In the case of purely bipolar pulsed field delivery, distal portion 20 includes electrodes that form the bipolar configuration for energy delivery. In an alternate configuration, a plurality of the electrodes 24 may serve as one pole while a second device containing one or more electrodes (not pictured) would be placed to serve as the opposing pole of the bipolar configuration. For example, as shown in FIG. 1, the distal portion 20 may include an electrode carrier arm 22 that is transitionable between a linear configuration and an expanded configuration in which the carrier arm 22 has an arcuate or substantially circular configuration. The carrier arm 22 may include the plurality of electrodes 24 (for example, nine electrodes 24, as shown in FIG. 1) that are configured to deliver pulsed-field energy. Further, the carrier arm 22 when in the expanded configuration may lie in a plane that is substantially orthogonal to the longitudinal axis of the elongate body 16. The planar orientation of the expanded carrier arm 22 may facilitate ease of placement of the plurality of electrodes 24 in contact with the target tissue. Alternatively, the medical device 12 may be have a linear configuration with the plurality of electrodes 24. For example, the distal portion 20 may include six electrodes 24 linearly disposed along a common longitudinal axis.

The generator 14 may include processing circuitry including a first processor 17 in communication with one or more controllers and/or memories containing software modules containing instructions or algorithms to provide for the automated operation and performance of the features, sequences, calculations, or procedures described herein. The system 10 may further include three or more surface ECG electrodes 26 on the patient in communication with the generator 14 through the catheter electrode distribution box 13 to monitor the patient's cardiac activity. In addition to monitoring, recording or otherwise conveying measurements or conditions within the medical device 12 or the ambient environment at the distal portion of the medical device 12, additional measurements may be made through connections to the multi-electrode catheter including for example temperature, electrode-tissue interface impedance, delivered charge, current, power, voltage, work, or the like in the generator 14 and/or the medical device 12.

The surface ECG electrodes 26 may be in communication with the generator 14 for initiating or triggering one or more alerts or therapeutic deliveries during operation of the medical device 12. Additional neutral electrode patient ground patches (not pictured) may be employed to evaluate the desired bipolar electrical path impedance, as well as monitor and alert the operator upon detection of inappropriate and/or unsafe conditions, which include, for example, improper (either excessive or inadequate) delivery of charge, current, power, voltage and work performed by the plurality of electrodes 24; improper and/or excessive temperatures of the plurality of electrodes 24, improper electrode-tissue interface impedances; improper and/or inadvertent electrical connection to the patient prior to delivery of high voltage energy by delivering one or more low voltage test pulses to evaluate the integrity of the tissue electrical path.

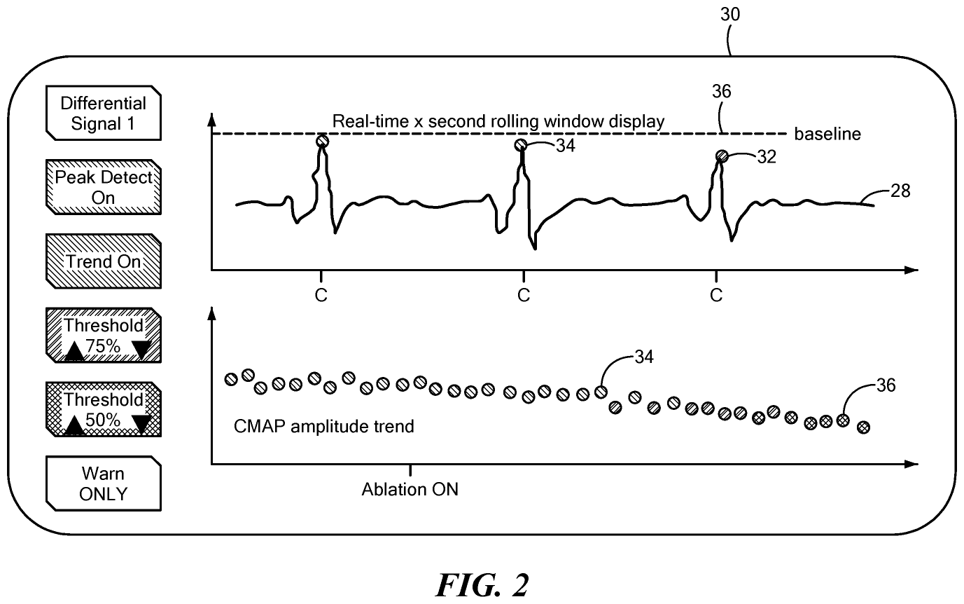

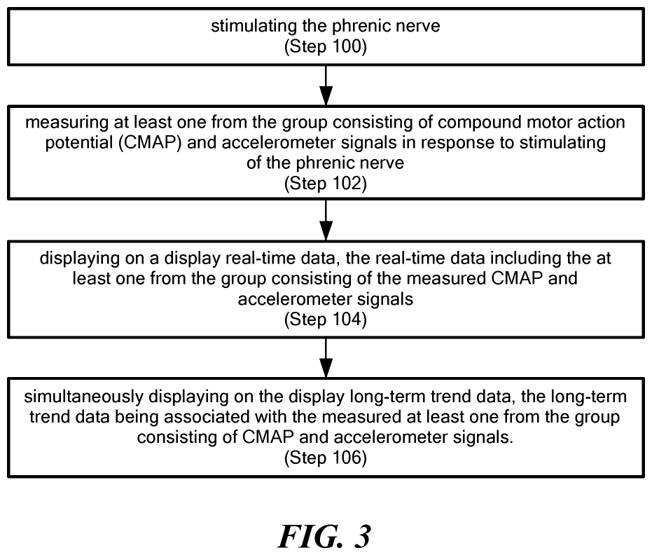

Referring now to FIGS. 2-3, during a cardiac ablation procedure using medical device 12 or a separate medical device, for example, a cryoablation device with a balloon or focal catheter, the phrenic nerve may be monitored for collateral damage and the extent thereof (Step 100). In particular, surface ECG electrodes 26 or other electrodes may be used to monitor and measure phrenic nerve activity, namely, compound motor action potential (CMAP) during the cardiac ablation procedure (Step 102). Optionally, or additionally, one or more accelerometers may be positioned on the patient's skin to monitor the phrenic nerve as a function of diaphragmatic movement. Initially, the phrenic nerve may be stimulated or paced with a separate medical device and CMAP or diaphragmatic movement is measured. Real-time data 28 which includes a CMAP or accelerometer signals is displayed on a display 30, which may be integral with controller 15 or a separate display, for example, on a console (Step 106). The real-time data 28 may be a direct reading of the CMAP or accelerometer signal. Feature extraction and filtering, such as peak signal detection or Fourier-transforms of the real-time data 28, may also be displayed in real-time for a predetermined period of time that allows sufficient display resolution, such as the last three cycles of accelerometer activation or provided phrenic nerve stimulation. Several potential functions or methods for analysis can be utilized, either singly or in combination with one another. Such feature extraction may include, peak detection, Fourier or wavelet content at particular frequencies or within certain frequency bands, CMAP signal morphology changes such as width, and/or delays between the initiating pacing pulse and the CMAP registered response. In addition, more sophisticated algorithms based upon intelligent algorithms such as Bayesian networks may be leveraged to more effectively discriminate between real, clinically interesting signals versus likely false positive trends. The real-time data 28 may be filtered by the processing circuitry before being displaying on the display 30.

As shown in FIG. 2, displaying of the real-time data 28 includes displaying the CMAP or accelerometer data in a rolling window. In one configuration, the rolling window includes the previous three phrenic nerve stimulations, but any time window, for example, 30 seconds, or number of previous phrenic nerve stimulations is contemplated as the rolling window. For example, as shown in FIG. 2, three phrenic nerve CMAP cycles are shown with the letter “C” denoting each cycle on the x-axis. In the configuration shown in FIG. 2, the real-time data 28 is superimposed with predetermined signal feature extraction markers 32. For example, the peak amplitude is extracted from each CMAP cycle and is displayed along with a real-time baseline peak CMAP 36 of the phrenic nerve function before the ablation cycle. In one configuration the predetermined signal feature extraction markers 32 are color coded and superimposed on the real-time data 28. For example, green, yellow, and red to indicate various levels of degradation of the peak CMAP 34. In one configuration, the predetermined signal feature extraction markers are correlated to a predetermined percentage threshold from peak amplitude. For example, yellow may be indicated of at least 50% reduction in peak CMAP 34 and red may be indicative of at least 75% reduction in peak CMAP 34, although any percentage is contemplated and the thresholds may be configured by the user.

In addition to displaying real-time data 28, the controller 15 and its processing circuitry is configured to further display long-term trend data 36 simultaneously with the real-time data 28 (Step 106). In the configuration shown in FIG. 2, the long-term trend data 36 is displayed beneath the real-time data 28 on the same display 30, although long-term trend data 36 may be displayed in any manner with respect to the real-time data 28. The long-term trend data 36 may include, but is not limited to, the peak CMAP 34 measured from each of the previous cycles of phrenic nerve stimulation. For example, while the real-time data 28 displays a rolling window, for example, the last three cycles of phrenic nerve stimulation, the long-term trend data 36 shows the trend of just the peak CMAP 34 over time. As shown in FIG. 2, in one configuration, each peak CMAP 34 point is displayed in color, and each color-coded peak CMAP 34 is correlated to a predetermined percentage threshold from a baseline peak amplitude. Optionally, a point at which the cardiac ablation procedure is initiated may also be displayed as part of the long-term trend data 36. The user viewing the real-time data 28 along with the long-term trend data 36 may also receive audible or visual alerts when a predetermined threshold is reached. For example, when the peak CMAP 34 exceeds a predetermined threshold, for example, from yellow to red in the color-coded scheme, an alert may be generated by the controller 15. Optionally, the controller 15 may be configured to automatically terminate or modify treatment of cardiac tissue if certain threshold criteria are met.

It will be appreciated by persons skilled in the art that the present invention is not limited to what has been particularly shown and described herein above. In addition, unless mention was made above to the contrary, it should be noted that all of the accompanying drawings are not to scale. A variety of modifications and variations are possible in light of the above teachings without departing from the scope and spirit of the invention, which is limited only by the following claims.

What is claimed is:

1. A method of monitoring a patient for phrenic nerve collateral damage during a cardiac ablation procedure, the method comprising:
stimulating the phrenic nerve;
measuring at least one from the group consisting of compound motor action potential (CMAP) and accelerometer signals in response to stimulating of the phrenic nerve;
displaying on a display real-time data, the real-time data including the at least one from the group consisting of the measured CMAP and accelerometer signals; and
simultaneously displaying on the display long-term trend data, the long-term trend data being associated with the measured at least one from the group consisting of CMAP and accelerometer signals.

2. The method of claim 1, wherein displaying the real-time data includes displaying a rolling window of the real time data.

3. The method of claim 2, wherein the rolling widow includes a predetermined number of previous cycles of phrenic nerve stimulation.

4. The method of claim 1, wherein the real-time data is filtered before it is displayed on the display.

5. The method of claim 1, wherein the real-time data is superimposed with predetermined signal feature extraction markers.

6. The method of claim 5, wherein the predetermined signal feature extraction markers are color coded.

7. The method of claim 5, wherein the predetermined signal feature extraction markers are correlated to a predetermined percentage threshold from peak amplitude.

8. The method of claim 1, wherein displaying the real-time data further includes displaying a pre-ablation baseline peak amplitude.

9. The method of claim 1, wherein simultaneously displaying the long-term trend data includes displaying a peak amplitude from each of a previous cycle of phrenic nerve stimulation.

10. The method of claim 9, wherein each peak amplitude is color coded.

11. The method of claim 10, wherein each color-coded peak amplitude is correlated to a predetermined percentage threshold from a baseline peak amplitude.

12. The method of claim 11, further including displaying with the long-term trend data a point at which the cardiac ablation procedure is initiated.

13. A method of monitoring a patient for phrenic nerve collateral damage during a cardiac ablation procedure, the method comprising:
stimulating the phrenic nerve;
measuring a compound motor action potential (CMAP) signal in response to the stimulation of the phrenic nerve;
displaying on a display, a rolling window of real-time data including a predetermined number of previous cycles of phrenic nerve stimulation, the real-time data including the measured CMAP signal; and
simultaneously displaying on the display long-term trend data, the long-term trend data being associated with the measured CMAP signal and including a peak amplitude from each of the previous cycles of phrenic nerve stimulation.

14. The method of claim 13, wherein the real-time data is superimposed with predetermined signal feature extraction markers.

15. The method of claim 14, wherein the predetermined signal feature extraction markers are color-coded.

16. The method of claim 14, wherein the predetermined signal feature extraction markers are correlated to a predetermined percentage threshold from peak amplitude.

17. The method of claim 13, wherein displaying the real-time data further includes displaying a pre-ablation baseline peak amplitude.

18. The method of claim 17, wherein each peak amplitude is color-coded, and wherein each color-coded peak amplitude is correlated to a predetermined percentage threshold from the pre-ablation baseline peak amplitude.

19. The method of claim 13, further including displaying with the long-term trend data a point at which the cardiac ablation procedure is initiated.

20. A method of monitoring a patient for phrenic nerve collateral damage during a cardiac ablation procedure, the method comprising:
stimulating the phrenic nerve;
measuring a compound motor action potential (CMAP) signal in response to the stimulation of the phrenic nerve;
displaying, on a display, a rolling window of real-time data including a predetermined number of previous cycles of phrenic nerve stimulation, the real time data including the measured CMAP signal and a pre-ablation baseline peak amplitude;
superimposing on the real-time data color coded predetermined signal feature extraction markers; and
simultaneously displaying on the display long term trend data, the long-term trend data being associated with the measured CMAP signal and including a peak amplitude from each of the previous cycles of phrenic nerve stimulation, each peak amplitude is color-coded, and each color-coded peak amplitude is correlated to a predetermined percentage threshold from the pre-ablation baseline peak amplitude.
